(19) United States
(12) Patent Application Publication
(10) Pub. No.: US 2011/0288466 A1
(43) Pub. Date: Nov. 24, 2011

(54) SYSTEMS AND METHODS FOR ACTIVATING CROSS-LINKING IN AN EYE

(75) Inventors: David Muller, Boston, MA (US); John Marshall, Farnborough (GB); Stephen L. Trokel, New York, NY (US); Peter Hersh, Far Hills, NJ (US); Pavel Kamaev, Lexington, MA (US)

(73) Assignee: AVEDRO, INC., Waltham, MA (US)

(21) Appl. No.: 13/086,019

(22) Filed: Apr. 13, 2011

Related U.S. Application Data
(60) Provisional application No. 61/323,388, filed on Apr. 13, 2010.
(60) Provisional application No. 61/326,527, filed on Apr. 21, 2010.
(60) Provisional application No. 61/345,873, filed on May 18, 2010.
(60) Provisional application No. 61/378,281, filed on Aug. 30, 2010.

(51) Int. Cl. A61M 37/00

(52) U.S. Cl. 604/20; 604/22; 604/521

(57) ABSTRACT

Devices and approaches for activating cross-linking within at least one eye component of an eye to stabilize and strengthen corneal tissue or other tissues of the eye. Cross-linking is activated within the at least one eye component by conveying a cross-linking agent to regions of the at least one eye component and then activating the cross-linking agent by delivering an initiating element to the at least one eye component. Approaches disclosed herein allow for precisely controlling the three dimensional region of strengthened tissue by conveying the cross-linking agent to regions of the at least one eye component. Approaches allow for conveying the cross-linking agent to a depth below the corneal surface such that cross-linking is activated below the corneal surface.

### CROSS REFERENCE TO RELATED APPLICATIONS

This application claims priority to: U.S. Provisional Application No. 61/323,388, filed Apr. 13, 2010; U.S. Provisional Application No. 61/326,527, filed Apr. 21, 2010; U.S. Provisional Application No. 61/345,873, filed May 18, 2010; U.S. Provisional Application No. 61/378,281, filed Aug. 30, 2010, the contents of each of these applications being incorporated entirely herein by reference.

### BACKGROUND

1. Field of the Invention

The disclosure pertains to systems and methods for stabilizing corneal tissue, and more particularly, systems and methods for conveying a cross-linking agent to regions of the cornea where the cross-linking agent is activated by an initiating element.

2. Description of Related Art

A variety of eye disorders, such as myopia, keratoconus, and hyperopia, involve abnormal shaping of the cornea. Laser-assisted in-situ keratomileusis (LASIK) is one of a number of corrective procedures that reshape the cornea so that light traveling through the cornea is properly focused onto the retina located in the back of the eye. During LASIK eye surgery, an instrument called a microkeratome is used to cut a thin flap in the cornea. The cornea is then peeled back and the underlying cornea tissue ablated to the desired shape with an excimer laser. After the desired reshaping of the cornea is achieved, the cornea flap is put back in place and the surgery is complete.

In another corrective procedure that reshapes the cornea, thermokeratoplasty provides a noninvasive procedure that applies electrical energy in the microwave or radio frequency (RF) band to the cornea. In particular, the electrical energy raises the corneal temperature until the collagen fibers in the cornea shrink at about 60° C. The onset of shrinkage is rapid, and stresses resulting from this shrinkage reshape the corneal surface. Thus, application of energy according to particular patterns, including, but not limited to, circular or annular patterns, may cause aspects of the cornea to flatten and improve vision in the eye.

The success of procedures, such as LASIK or thermokeratoplasty, in addressing eye disorders, such as myopia, keratoconus, and hyperopia, depends on the stability of the changes in the corneal structure after the procedures have been applied.

### BRIEF SUMMARY

According to aspects of the present disclosure provide systems and methods for stabilizing corneal tissue and improving its biomechanical strength, particularly after desired structural changes have been achieved in the corneal tissue. For example, the embodiments help to preserve the desired reshaping of the cornea produced by LASIK surgery, thermokeratoplasty, or other similar treatments.

According to aspects of the present disclosure, after a treatment produces a desired change to the shape of a cornea, a cross-linking agent is activated in the treated region of the cornea. The cross-linking agent prevents the corneal fibrils in the treated regions from moving and causing undesired changes to the shape of the cornea. An initiating element may be applied to the treated corneal fibrils to activate the cross-linking agent.

In some embodiments, for example, the cross-linking agent may be Riboflavin and the initiating element may be photoactivating light, such as ultraviolet (UV) light. In these embodiments, the photoactivating light initiates cross-linking activity by irradiating the applied cross-linking agent to release reactive oxygen radicals in the corneal tissue. In particular, the cross-linking agent, e.g., Riboflavin, acts as a sensitizer to convert O₂ into singlet oxygen which causes cross-linking within the corneal tissue.

The initiating element may be applied according to a selected pattern to stabilize and strengthen the regions of the cornea where structural changes have been generated by the treatment. Accordingly, aspects of the present disclosure may include a delivery system that accurately and precisely delivers the initiating element to corneal fibrils according to a selected pattern. In embodiments where the initiating element is UV light, the delivery system may deliver the UV light in the form of a laser.

According to an aspect of the present disclosure a method is disclosed for activating cross-linking in at least one eye component. The method includes delivering ultrasound waves to the at least one eye component such that a permeability of the at least one eye component to the cross-linking agent is increased. The method also includes conveying the cross-linking agent to the at least one eye component. The method also includes activating the cross-linking agent by delivering an initiating element to the at least one eye component.

According to another aspect of the present disclosure a method is disclosed for treating an eye. The method includes: delivering heat energy to at least one eye component of the eye; generating a structural change in the at least one eye component; and lowering an intraocular pressure of the eye due to the generated structural change.

According to another aspect of the present disclosure a method is disclosed for activating cross-linking in at least one eye component. The method includes delivering heat energy to the at least one eye component such that a permeability of the at least one eye component to a cross-linking agent is increased. The method also includes conveying a cross-linking agent to the at least one eye component. The method also includes activating the cross-linking agent by delivering an initiating element to the at least one eye component.

According to yet another aspect of the present disclosure a method is disclosed for activating cross-linking in at least one eye component. The method includes conveying a charged cross-linking agent to the at least one eye component. The method also includes urging the charged cross-linking agent to a depth of the at least one eye component using iontophoresis. The method also includes activating the cross-linking agent by delivering an initiating element to the at least one eye component.

According to still another aspect of the present disclosure a method is disclosed for activating cross-linking in at least one eye component of an eye. The method includes conveying the cross-linking agent to a surface of the at least one eye component. The method also includes allowing a time to pass to allow the cross-linking agent to diffuse within the at least one eye component. The method also includes applying a reverse osmotic fluid to the surface of the at least one eye component to draw out the cross-linking agent at or near the surface of the at least one eye component. The method also includes activating the cross-linking agent by delivering an initiating element to the at least one eye component.

These and other aspects of the present disclosure will become more apparent from the following detailed description of embodiments of the present disclosure when viewed in conjunction with the accompanying drawings.

### BRIEF DESCRIPTION OF THE DRAWINGS

FIG. 1 provides a block diagram of an example delivery system for delivering a cross-linking agent and an activator to a cornea of an eye in order to initiate molecular cross-linking of corneal collagen within the cornea.

FIG. 2A provides a flowchart showing an example embodiment according to aspects of the present disclosure for activating cross-linking within cornea tissue using a cross-linking agent and an initiating element.

FIG. 2B provides a flowchart similar to FIG. 2A where Riboflavin may be applied topically as the cross-linking agent and UV light may be applied as the initiating element.

FIG. 3 illustrates an example system for applying energy to a cornea of an eye to generate heat and cause reshaping of the cornea.

FIG. 4A illustrates a high resolution image of a cornea after energy has been applied.

FIG. 4B illustrates another high resolution image of the cornea of FIG. 4A.

FIG. 4C illustrates a histology image of the cornea of FIG. 4A.

FIG. 4D illustrates another histology image of the cornea of FIG. 4A.

FIG. 5A illustrates an example embodiment of an approach for stabilizing a change in corneal structure by activating cross-linking following thermokeratoplasty.

FIG. 5B illustrates an example embodiment of an approach for stabilizing a change in corneal structure by activating cross-linking following LASIK surgery.

FIG. 6A illustrates an example embodiment of an approach for stabilizing corneal structure by conveying a cross-linking agent to regions of a cornea using ultrasound to increase the permeability thereof.

FIG. 6B illustrates an example embodiment similar to FIG. 6A where microspheres are utilized in combination with the ultrasound to enhance the permeability of the corneal tissue.

FIG. 7A illustrates an example embodiment of an approach for stabilizing corneal structure by conveying a cross-linking agent to regions of a cornea using iontophoresis.

FIG. 7B illustrates an example embodiment similar to FIG. 7A where the cross-linking agent is dissolved in a solvent to enhance its permeability.

FIG. 8A illustrates an example embodiment of an approach for stabilizing corneal structure by conveying a cross-linking agent to regions of a cornea using a reverse osmotic fluid to draw the cross-linking agent away from the corneal surface.

FIG. 8B illustrates an example embodiment of an approach for stabilizing corneal structure by conveying a cross-linking agent to regions of a cornea using a neutral compound to increase pressure on the cross-linking agent.

FIG. 9A illustrates an example embodiment of an approach for stabilizing corneal structure by allowing a cross-linking agent to diffuse through the cornea during a period of time T.

FIG. 9B illustrates an example embodiment similar to FIG. 9A where the cross-linking agent is Riboflavin and the initiating element is UV light.

FIG. 10 illustrates an example embodiment of an approach for conveying a cross-linking agent to a region of a cornea by using a combination of one or more approaches discussed herein.

### DETAILED DESCRIPTION

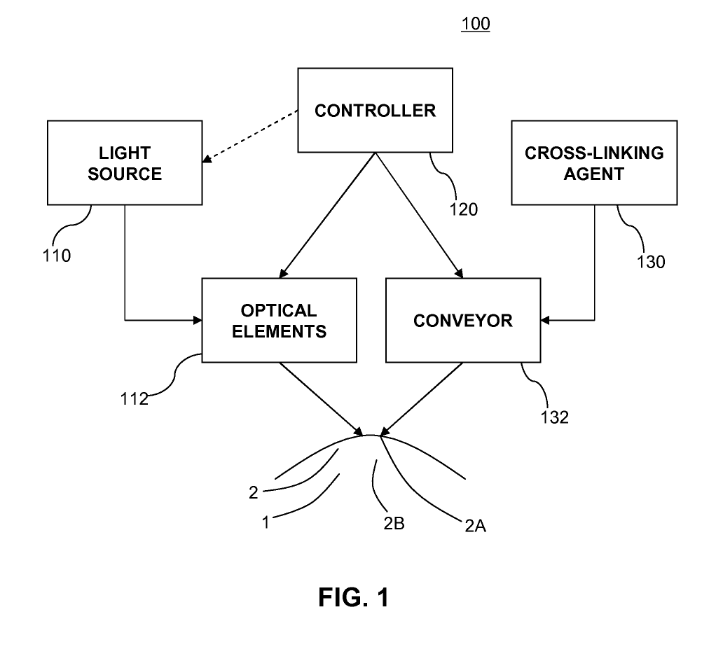

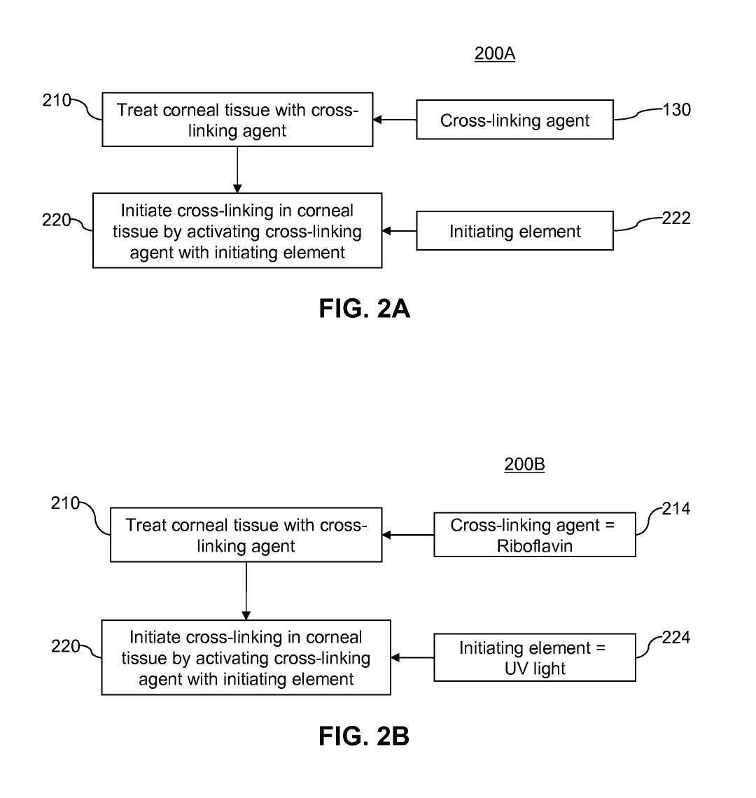

FIG. 1 provides a block diagram of an example delivery system 100 for delivering a cross-linking agent 130 and an initiating element (e.g. 222 shown in FIG. 2A) to a cornea 2 of an eye 1 in order to initiate molecular cross-linking of corneal collagen fibrils within the cornea 2. Cross-linking can stabilize corneal tissue and improve its biomechanical strength. The delivery system 100 includes a conveyor 132 for conveying the cross-linking agent 130 to the cornea 2. The cross-linking agent 130 can be conveyed to the cornea 2 by, for example, an eye dropper, a drug application device, such as a bandage, or can be included in a cream applied to the eye 1. The delivery system 100 includes a light source 110 and optical elements 112 for directing light to the cornea 2. The delivery system 100 also includes a controller 120 that is coupled to the conveyor 132 and the optical elements 112. The conveyor 132 may be an apparatus adapted to apply the cross-linking agent 130 according to particular patterns on the cornea 2 advantageous for causing cross-linking to take place within the corneal tissues. The conveyor 132 may apply the cross-linking agent 130 to a corneal surface 2A (e.g., an epithelium), or to other locations on the eye 1. Particularly, the conveyor 132 may apply the cross-linking agent 130 to an abrasion or cut of the corneal surface 2A to facilitate the transport or penetration of the cross-linking agent through the cornea 2 to a mid-depth region 2B. As described further herein, the conveyor 132 can be adapted to convey the cross-linking agent 130 to regions of the eye 1 including depths below the corneal surface 2A, by utilizing iontophoresis, reverse-osmotic fluids, neutral compounds, and/or by increasing the permeability of the cornea 2 using heat energy and/or ultrasound to modify the corneal structure and increase its permeability. Additionally or alternatively, the delivery system 100 can include one or more additional components (not shown) to convey the cross-linking agent 130.

For the sake of clarity, the non-limiting embodiments disclosed herein describe the operation of aspects of the present disclosure in conveying the cross-linking agent 130 to regions of the cornea 2. However, the system 100, and the embodiments disclosed herein for conveying the cross-lining agent 130 to regions of the eye 1, refer to systems and methods for directing the cross-linking agent 130 to regions of at least one eye component, and it is understood that the at least one eye component may include a cornea, a limbus, a sclera, and/or a retina.

As described below in connection with FIGS. 2A-2B, which describe an exemplary operation of the delivery system 100, the cross-linking agent 130 is applied to the cornea 2 using the conveyor 132. Once the cross-linking agent 130 has been applied to the cornea 2, the cross-linking agent 130 is initiated by the light source 110 (i.e. the initiating element) to cause cross-linking agent 130 to absorb enough energy to release free oxygen radicals within the cornea 2. Once released, the free oxygen radicals (i.e. singlet oxygen) form covalent bonds between corneal collagen fibrils and thereby cause the corneal collagen fibrils to cross-link and change the structure of the cornea 2. For example, activation of the cross-linking agent 130 with the light source 110 delivered to the cornea 2 through the optical elements 112 may result in cross-linking in the mid-depth region 2B of the cornea 2 and thereby strengthen and stiffen the structure of the cornea 2.

Although eye therapy treatments may initially achieve desired reshaping of the cornea 2, the desired effects of reshaping the cornea 2 may be mitigated or reversed at least partially if the collagen fibrils within the cornea 2 continue to change after the desired reshaping has been achieved. Indeed, complications may result from further changes to the cornea 2 after treatment. For example, a complication known as post-LASIK ectasia may occur due to the permanent thinning and weakening of the cornea 2 caused by LASIK surgery. In post-LASIK ectasia, the cornea 2 experiences progressive steepening (bulging).

Aspects of the present disclosure provide approaches for initiating molecular cross-linking of corneal collagen to stabilize corneal tissue and improve its biomechanical strength. For example, embodiments may provide devices and approaches for preserving the desired corneal structure and shape that result from an eye therapy treatment, such as LASIK surgery or thermokeratoplasty. In addition, aspects of the present disclosure may provide devices and approaches for initiating cross-linking at depths of the cornea 2 below the epithelium 2A by first conveying the cross-linking agent 130 to regions of the cornea 2 below the epithelium 2A. Advantageously, aspects of the present disclosure allow for conveying the cross-linking agent 130 to regions below the epithelium 2A of the cornea 2 without requiring removal of the epithelium 2A. As described herein, the devices and approaches disclosed herein may be used to preserve desired shape or structural changes following an eye therapy treatment by stabilizing the corneal tissue of the cornea 2. The devices and approaches disclosed herein may also be used to enhance the strength or biomechanical structural integrity of the corneal tissue apart from any eye therapy treatment.

Some approaches initiate molecular cross-linking in a treatment zone of the cornea 2 where structural changes have been induced by, for example, LASIK surgery or thermokeratoplasty. However, it has been discovered that initiating cross-linking directly in this treatment zone may result in undesired haze formation. Accordingly, aspects of the present disclosure also provide alternative techniques for initiating cross-linking to minimize haze formation. In particular, the structural changes in the cornea 2 are stabilized by initiating cross-linking in selected areas of corneal collagen outside of the treatment zone. This cross-linking strengthens corneal tissue neighboring the treatment zone to support and stabilize the actual structural changes within the treatment zone.

With reference to FIG. 1, the optical elements 112 may include one or more mirrors or lenses for directing and focusing the light emitted by the light source 110 to a particular pattern on the cornea 2 suitable for activating the cross-linking agent 130. The light source 110 may be an ultraviolet light source, and the light directed to the cornea 2 through the optical elements 112 may be an activator of the cross-linking agent 130. The light source 110 may also alternatively or additionally emit photons with greater or lesser energy levels than ultraviolet light photons. The light source 110 can be a laser light source, and may emit a slightly diverging beam of nearly collimated light. For eye safety reasons, it is desirable that implementations incorporating a laser light source as the light source 110 deliver a slightly diverging beam, and prevent the occurrence of a converging beam being applied to the eye 1. Implementations utilizing a laser light source as the light source 110 are desirably able to deliver light emitted by the light source 110 to the eye 1 at an intensity level that is largely insensitive to changes in a distance from the light source 110.

The delivery system 100 also includes a controller 120 for controlling the operation of the optical elements 112 or the conveyor 132, or both. By controlling aspects of the operation of the optical elements 112 and the conveyor 132, the controller 120 can control the regions of the cornea 2 that receive the cross-linking agent 130 and that are exposed to the light source 110. By controlling the regions of the cornea 2 that receive the cross-linking agent 130 and the light source 110, the controller 120 can control the particular regions of the cornea 2 that are strengthened and stabilized through cross-linking of the corneal collagen fibrils. Furthermore, by controlling the depth region that the cross-linking agent 130 is conveyed to, the controller 120 can control the depth of cross-linking activity within the corneal tissue. In an implementation, the cross-linking agent 130 can be applied generally to the eye 1, without regard to a particular region of the cornea 2 requiring strengthening, but the light source 110 can be directed to a particular region of the cornea 2 requiring strengthening, and thereby control the region(s) of the cornea 2 wherein cross-linking is initiated by controlling the regions of the cornea 2 that are exposed to the light source 110. In another implementation, the light source 110 can be directed generally to the eye 1, and the cross-linking agent 130 can be conveyed to particular region(s) of the cornea 2, and thereby control the region of the cornea 2 wherein cross-linking is initiated. In yet another implementation, both the cross-linking agent 130 and the initiating element 110 can be conveyed and/or directed to particular regions of the cornea 2, and thereby jointly control the region(s) of the cornea 2 wherein cross-linking is initiated.

The optical elements 112 can be used to focus the light emitted by the light source 110 to a particular focal plane within the cornea 2, such as a focal plane that includes the mid-depth region 2B. In addition, according to particular embodiments, the optical elements 112 may include one or more beam splitters for dividing a beam of light emitted by the light source 110, and may include one or more heat sinks for absorbing light emitted by the light source 110. The optical elements 112 may further include filters for partially blocking wavelengths of light emitted by the light source 110 and for advantageously selecting particular wavelengths of light to be directed to the cornea 2 for activating the cross-linking agent 130. The controller 120 can also be adapted to control the light source 110 by, for example, toggling a power switch of the light source 110.

In an implementation, the controller 120 may include hardware and/or software elements, and may be a computer. The controller 120 may include a processor, a memory storage, a microcontroller, digital logic elements, software running on a computer processor, or any combination thereof. In an alternative implementation of the delivery system 100, the controller 120 may be replaced by two or more separate controllers or processors. For example, one controller may be used to control the operation of the conveyor 132, and thereby control the precise rate of delivery, location of conveyance, depth of penetration, and/or concentration of the cross-linking agent 130 to the cornea 2. Another controller may be used to control the operation of the optical elements 112, and thereby control with precision the delivery of the light source 110 (i.e. the initiating element) to the cornea 2 by controlling any combination of wavelength, bandwidth, intensity, power, location, depth of penetration, and duration of treatment. In addition, the function of the controller 120 can be partially or wholly replaced by a manual operation. For example, the conveyor 132 can be manually operated to deliver the cross-linking agent 130 to the cornea 2 without the assistance of the controller 120. In addition, the controller 120 can operate the conveyor 132 and the optical elements 112 according to inputs dynamically supplied by an operator of the delivery system 100 in real time, or can operate according to a pre-programmed sequence or routine.

Referring to FIG. 2A, an example embodiment 200A according to aspects of the present disclosure is illustrated. Specifically, in step 210, the corneal tissue is treated with the cross-linking agent 130. Step 210 may occur, for example, after a treatment is applied to generate structural changes in the cornea and produce a desired shape change. Alternatively, step 210 may occur, for example, after it has been determined that the corneal tissue requires stabilization or strengthening. The cross-linking agent 130 is then activated in step 220 with an initiating element 222. In an example configuration, the initiating element 222 may be the light delivered from the light source 110 shown in FIG. 1. Activation of the cross-linking agent 130, for example, may be triggered thermally by the application of microwaves or light.

As the example embodiment 200B of FIG. 2B shows further, Riboflavin may be applied topically as a cross-linking agent 214 to the corneal tissue in step 210. As also shown in FIG. 2B, ultraviolet (UV) light may be applied as an initiating element 224 in step 220 to initiate cross-linking in the corneal areas treated with Riboflavin. Specifically, the UV light initiates cross-linking activity by causing the applied Riboflavin to release reactive oxygen radicals in the corneal tissue. In particular, the Riboflavin acts as a sensitizer to convert O₂ into singlet oxygen which causes cross-linking within the corneal tissue.

According to one approach, the Riboflavin may be applied topically to the corneal surface, and transepithelial delivery allows the Riboflavin to be applied to the corneal stroma. In general, the application of the cross-linking agent sufficiently introduces Riboflavin to mid-depth regions of the corneal tissue where stronger and more stable structure is desired.

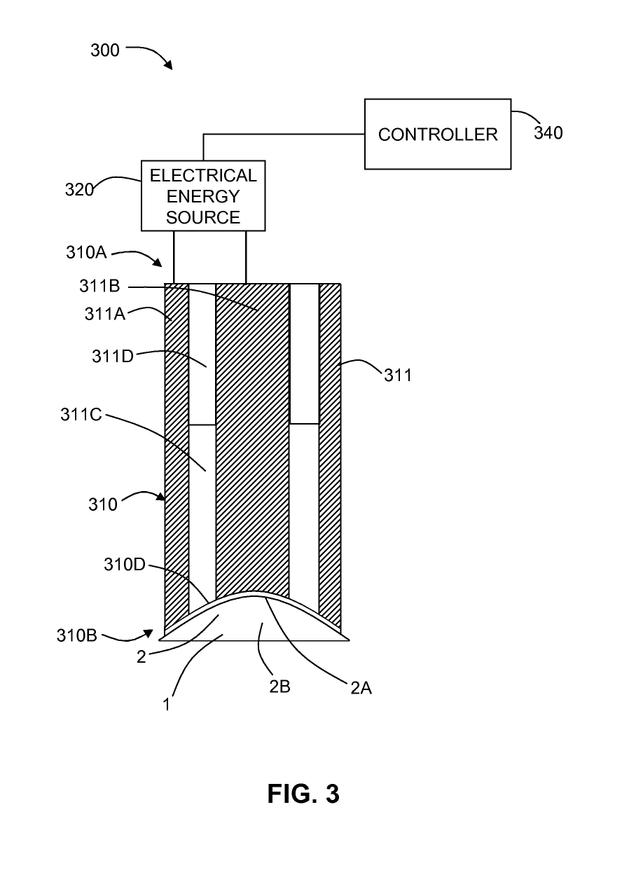

According to aspects of the present disclosure, a treatment is employed to produce a desired change to the shape of the cornea 2. For example, thermokeratoplasty applies energy to the cornea 2 to reshape the cornea 2. FIG. 3 illustrates an example system 300 for applying energy to a cornea 2 of an eye 1 to generate heat and cause reshaping of the cornea 2. In particular, FIG. 3 shows an applicator 310 with an electrical energy conducting element 311 that is operably connected to an electrical energy source 320, for example, via conventional conducting cables. The electrical energy conducting element 311 extends from a proximal end 310A to a distal end 310B of the applicator 310. The electrical energy conducting element 311 conducts electrical energy from the source 320 to the distal end 310B to apply energy to the cornea 2, which is positioned at the distal end 310B. In particular, the electrical energy source 320 may include a microwave oscillator for generating microwave energy. For example, the oscillator may operate at a microwave frequency range of 400 MHz to 3000 MHz, and more specifically at a frequency of around 915 MHz or 2450 MHz which has been safely used in other applications. As used herein, the term “microwave” may generally correspond to a frequency range from about 10 MHz to about 10 GHz.

As further illustrated in FIG. 3, the electrical energy conducting element 311 may include two microwave conductors 311A and 311B, which extend from the proximal end 310A to the distal end 310B of the applicator 310. In particular, the conductor 311A may be a substantially cylindrical outer conductor, while the conductor 311B may be a substantially cylindrical inner conductor that extends through an inner passage extending through the conductor 311A. With the inner passage, the conductor 311A has a substantially tubular shape. The inner and the outer conductors 311A and 311B may be formed, for example, of aluminum, stainless steel, brass, copper, other metals, coated metals, metal-coated plastic, or any other suitable conductive material.

With the concentric arrangement of conductors 311A and 311B, a substantially annular gap 311C of a selected distance is defined between the conductors 311A and 311B. The annular gap 311C extends from the proximal end 310A to the distal end 310B. A dielectric material 311D may be used in portions of the annular gap 311C to separate the conductors 311A and 311B. The distance of the annular gap 311C between conductors 311A and 311B determines at least partially the penetration depth of microwave energy into the cornea 2 according to established microwave field theory. Thus, the energy conducting element 311 receives, at the proximal end 310A, the electrical energy generated by the electrical energy source 320, and directs microwave energy to the distal end 311B, where the cornea 2 is positioned.

In general, the outer diameter of the inner conductor 311B may be selected to achieve an appropriate change in corneal shape, i.e., keratometry, induced by the exposure to microwave energy. Meanwhile, the inner diameter of the outer conductor 311A may be selected to achieve a desired gap between the conductors 311A and 311B. For example, the outer diameter of the inner conductor 311B ranges from about 2 mm to about 10 mm while the inner diameter of the outer conductor 311A ranges from about 2.1 mm to about 12 mm. In some systems, the annular gap 311C may be sufficiently small, e.g., in a range of about 0.1 mm to about 2.0 mm, to minimize exposure of the endothelial layer of the cornea (posterior surface) to elevated temperatures during the application of energy by the applicator 310.

A controller 340 may be employed to selectively apply the energy any number of times according to any predetermined or calculated sequence. In addition, the heat may be applied for any length of time. Furthermore, the magnitude of heat being applied may also be varied. Adjusting such parameters for the application of heat determines the extent of changes that are brought about within the cornea 2. Of course, the system 300 can limit the changes in the cornea 2 to an appropriate amount of shrinkage of collagen fibrils in a selected region and according to a selected pattern. When employing microwave energy to generate heat in the cornea 2, for example with the applicator 310, the microwave energy may be applied with low power (of the order of 40 W) and in long pulse lengths (of the order of one second). However, other systems may apply the microwave energy in short pulses. In particular, it may be advantageous to apply the microwave energy with durations that are shorter than the thermal diffusion time in the cornea 2. For example, the microwave energy may be applied in pulses having higher power in the range of 500 W to 3 kW and pulse duration in the range of about 10 milliseconds to about one second.

Referring again to FIG. 3, at least a portion of each of the conductors 311A and 311B may be covered with an electrical insulator to minimize the concentration of electrical current in the area of contact between the corneal surface (epithelium) 2A and the conductors 311A and 311B. In some systems, the conductors 311A and 311B, or at least a portion thereof, may be coated with a material that can function both as an electrical insulator as well as a thermal conductor. A dielectric layer 310D may be employed along the distal end 311B of the applicator 310 to protect the cornea 2 from electrical conduction current that would otherwise flow into the cornea 2 via conductors 311A and 311B. Such current flow may cause unwanted temperature effects in the cornea 2 and interfere with achieving a maximum temperature within the collagen fibrils in a mid-depth region 2B of the cornea 2. Accordingly, the dielectric layer 310D is positioned between the conductors 311A and 311B and the cornea 2. The dielectric layer 110D may be sufficiently thin to minimize interference with microwave emissions and thick enough to prevent superficial deposition of electrical energy by flow of conduction current. For example, the dielectric layer 310D may be a biocompatible material deposited to a thickness of between about 10 and 100 micrometers, preferably about 50 micrometers. As another example, the dielectric layer 310D can be a flexible sheath-like structure of biocompatible material that covers the conductors 311A and 311B at the distal end 310B and extends over a portion of the exterior wall of the outer conductor 311B. As still a further example, the dielectric layer 310D can include a first flexible sheath-like structure of biocompatible material that covers the distal end of the inner conductor 311A and a second flexible sheath-like structure of biocompatible material that covers the distal end of the outer conductor 311B.

In general, an interposing layer, such as the dielectric layer 310D, may be employed between the conductors 311A and 311B and the cornea 2 as long as the interposing layer does not substantially interfere with the strength and penetration of the microwave radiation field in the cornea 2 and does not prevent sufficient penetration of the microwave field and generation of a desired heating pattern in the cornea 2. The dielectric material may be elastic, such as polyurethane and silastic, or nonelastic, such as Teflon® and polyimides. The dielectric material may have a fixed dielectric constant or varying dielectric constant by mixing materials or doping the sheet, the variable dielectric being spatially distributed so that it may affect the microwave hearing pattern in a customized way. The thermal conductivity of the material may have fixed thermal properties (thermal conductivity or specific heat), or may also vary spatially, through mixing of materials or doping, and thus provide a means to alter the heating pattern in a prescribed manner. Another approach for spatially changing the heating pattern is to make the dielectric sheet material of variable thickness. The thicker region will heat less than the thinner region and provides a further means of spatial distribution of microwave heating.

During operation, the distal end 310B of the applicator 310 as shown in FIG. 3 is positioned on or near the corneal surface 2A. Preferably, the applicator 310 makes direct contact with the corneal surface 2A. In particular, such direct contact positions the conductors 311A and 311B at the corneal surface 2A (or substantially near the corneal surface 2A if there is a thin interposing layer between the conductors 311A and 311B and the corneal surface 2A). Accordingly, direct contact helps ensure that the pattern of microwave heating in the corneal tissue has substantially the same shape and dimension as the gap 311C between the two microwave conductors 311A and 311B.

The system 300 of FIG. 3 is provided for illustrative purposes only, and other systems may be employed to apply heat to cause reshaping of the cornea by causing rearrangement of the corneal collagen fibrils. Other systems are described, for example, in U.S. patent application Ser. No. 12/208,963, filed Sep. 11, 2008, which is a continuation-in-part application of U.S. patent application Ser. No. 11/898,189, filed on Sep. 10, 2007, the contents of these applications being entirely incorporated herein by reference.

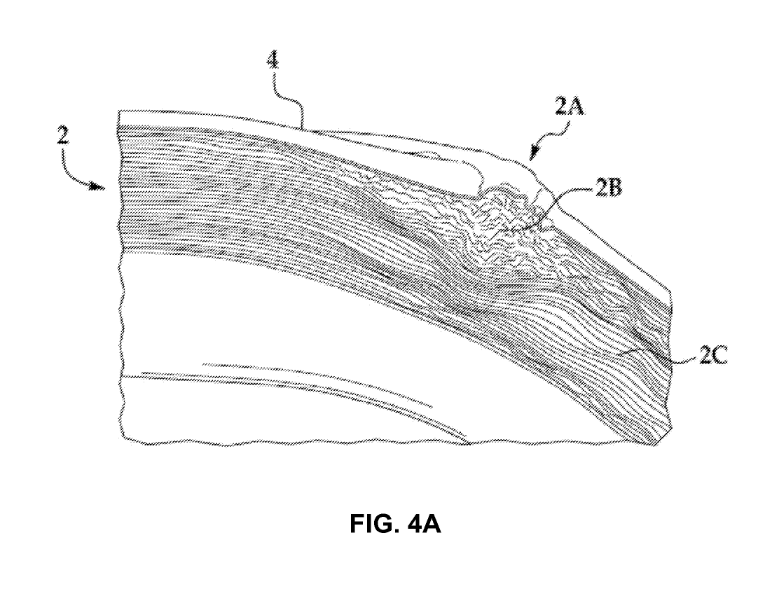

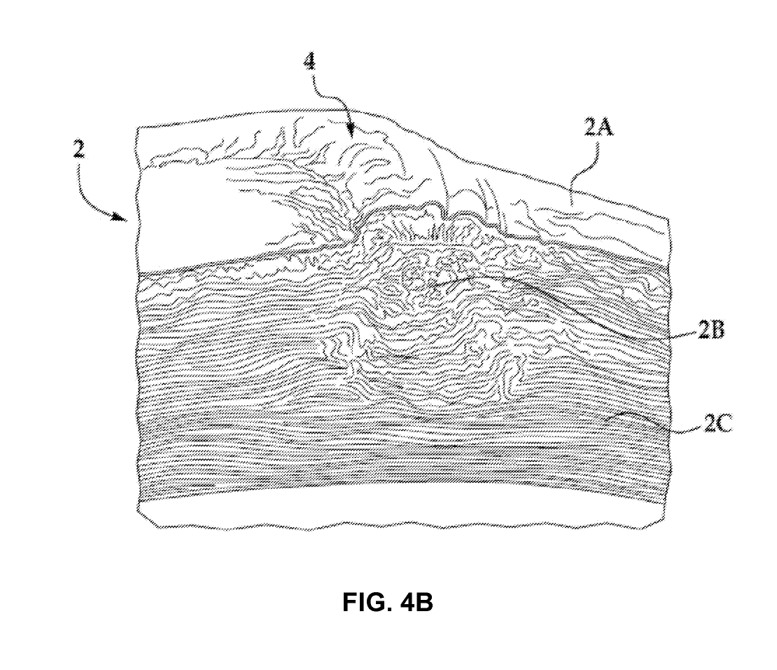

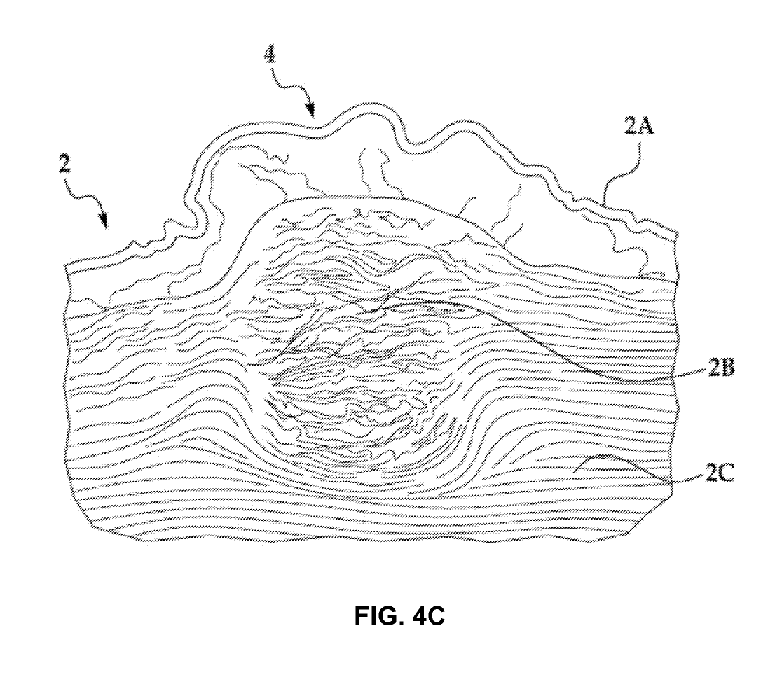

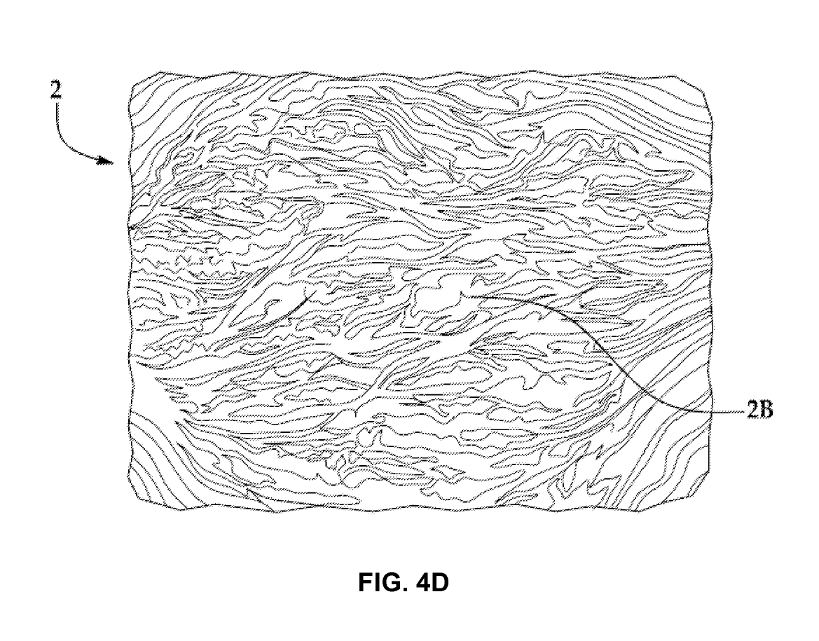

FIGS. 4A-D illustrate an example of the effect of applying heat to corneal tissue with a system for applying heat, such as the system 300 illustrated in FIG. 3. In particular, FIGS. 4A and 4B illustrate high resolution images of cornea 2 after heat has been applied. As FIGS. 4A and 4B show, a lesion 4 extends from the corneal surface 2A to a mid-depth region 2B in the corneal stroma 2C. The lesion 4 is the result of changes in corneal structure induced by the application of heat as described above. These changes in structure result in an overall reshaping of the cornea 2. It is noted that the application of heat, however, has not resulted in any heat-related damage to the corneal tissue.

As further illustrated in FIGS. 4A and 4B, the changes in corneal structure are localized and limited to an area and a depth specifically determined by an applicator as described above. FIGS. 4C and 4D illustrate histology images in which the tissue shown in FIGS. 4A and 4B has been stained to highlight the structural changes induced by the heat. In particular, the difference between the structure of collagen fibrils in the mid-depth region 2B where heat has penetrated and the structure of collagen fibrils outside the region 2B is clearly visible. Thus, the collagen fibrils outside the region 2B remain generally unaffected by the application of heat, while the collagen fibrils inside the region 2B have been rearranged and formed new bonds to create completely different structures. In other words, unlike processes, such as orthokeratology, which compress areas of the cornea to reshape the cornea via mechanical deformation, the collagen fibrils in the region 2B are in an entirely new state.

Treatment of the cornea 2 produces structural changes to the stroma 2C. As described previously with reference to FIGS. 4A-D, for example, the lesion 4 extends from the corneal surface 2A to a mid-depth region 2B in the corneal stroma 2C. In such cases, the application of the cross-linking agent (e.g. in step 210 of FIG. 2A-B) must introduce sufficient amounts of the cross-linking agent 130 to mid-depth regions of the corneal tissue where stronger and more stable structure is required. The epithelium 2A, however, may act as a barrier to the effective delivery of the cross-linking agent 130 to the stroma 2C. According to one technique, at least a portion of the epithelium 2A is removed prior to conveying the cross-linking agent 130 topically to the corneal stroma 2C. Removal of the epithelium 2A, however, may require a healing period, which may be accompanied by post-operative pain and other complications.

Thus, aspects of the present disclosure provide techniques that promote the effective delivery of the cross-linking agent 130 across the epithelium 2A to the corneal tissue below, without requiring removal of the overlying epithelium 2A.

Studies have been directed at the effects of conveying a cross-linking agent to a cornea after thermokeratoplasty. The studies have discovered distinct enhancement in the uptake, i.e., movement, of the cross-linking agent (Riboflavin) into the regions that are treated with energy. In particular, fluorescence indicating the presence of the cross-linking agent in the cornea is brighter in the regions that receive energy. In other words, the pattern of brighter fluorescence matches the pattern of energy application. The pattern of energy application generally depends on the shape of the thermokeratoplasty applicator and the contact between the applicator and the cornea. For example, using the applicator 310, the regions of the cornea exhibiting an enhanced capacity to receive the cross-linking agent correspond closely to the annular pattern defined by the outer conductor 311A and inner conductor 311B. In addition, the fluorescence is bigger and brighter when the intended correction via thermokeratoplasty is greater, i.e., greater amounts of energy are applied. Correspondingly, the greater uptake of the cross-linking agent in these treated regions should result in greater cross-linking when the cross-linking agent is appropriately activated. According to the studies, the delivery of the cross-linking agent is enhanced where cross-linking activation is particularly desired, i.e., where the energy is applied to treat the eye according to thermokeratoplasty.

As discussed previously with reference to FIGS. 4A-D, the application of the energy to the cornea during thermokeratoplasty changes the structure of the corneal tissue. As shown particularly in FIGS. 4C-D, the spacing between corneal fibrils increases after the energy is applied. This increased spacing, or permeability, enhances the movement of the cross-linking agent into the treated regions, because a greater volume of cross-linking agent can be received into the spacing. The studies indicate that energy may be applied to eye tissue, such as corneal tissue, to increase permeability. In general, the increased permeability may be advantageous in the treatment of a variety of disorders, such as abnormal shaping of the cornea, retinal membrane problems, or glaucoma.

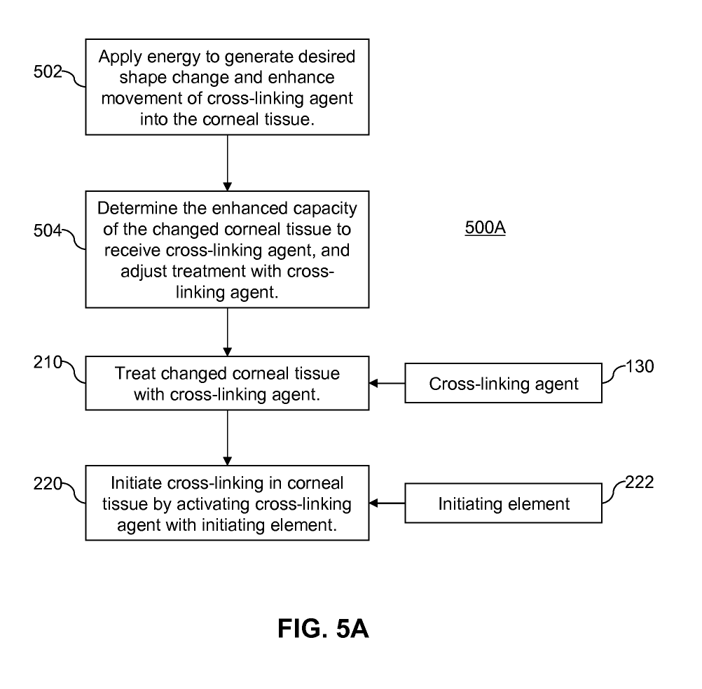

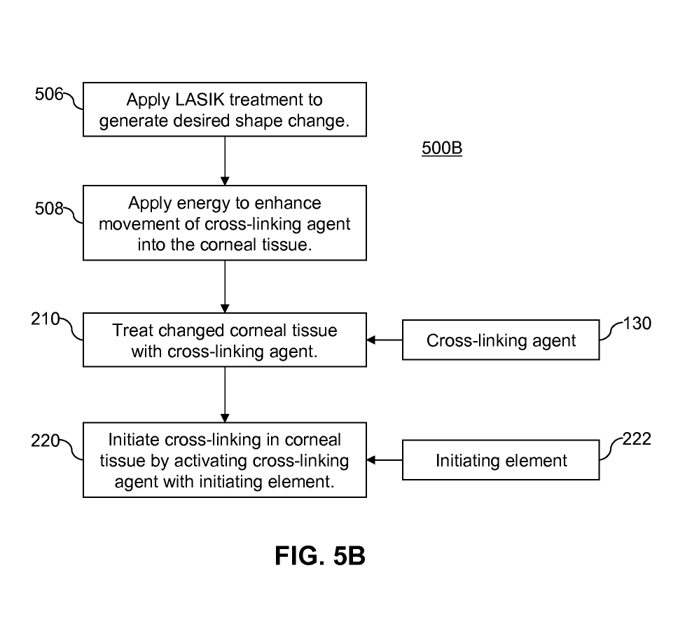

In some embodiments, the dosage of the cross-linking agent and other aspects of its application are modified to account for the increased capacity of treated regions to accommodate greater amounts of cross-linking agent after the application of energy. For example, referring to the example embodiment 500A shown in FIG. 5A, after thermokeratoplasty is applied in step 502, the enhanced capacity of the treated regions is determined and the dosage of the cross-linking agent 130 is correspondingly adjusted in step 504 before the cross-linking agent 130 is applied in step 210.

In other embodiments, another treatment, such as LASIK, may be employed. In LASIK surgery, an instrument called a microkeratome is used to cut a thin flap in the cornea. The flap is peeled back and the underlying corneal tissue is ablated to the desired shape with an excimer laser. After the desired reshaping of the cornea is achieved, the cornea flap is put back in place to complete the surgery. In such treatments, energy may not be applied to the cornea in the same manner as thermokeratoplasty. However, the application of energy may be employed as an additional step to enhance the movement of the cross-linking agent 130 into regions that are treated. For example, referring to the example embodiment 500B shown in FIG. 5B, after LASIK treatment is applied in step 506, an amount of energy is applied to the treated regions in step 508 to enhance the movement of the cross-linking agent into the corneal tissue in the treated regions. It is noted that an amount of energy may be applied so that the results of the treatment in step 506 are not significantly affected by the change in corneal structure resulting from the application of energy in step 508.

In general, energy may be applied to selected regions of the cornea 2 according to any pattern, for example, with an applicator similar to the applicator 310 described previously. Although the pattern defined by the applicator 310 may be annular, the pattern may have any non-annular and/or asymmetric shape. The pattern determines the regions of the cornea 2 that will have enhanced permeability to receive the cross-linking agent 130 and that will experience greater cross-linking relative to the other regions of the cornea 2. Thus, the application of energy provides a technique for achieving patterned activation of cross-linking in the cornea 2. Examples of the non-annular shapes by which energy may be applied to the cornea are described in U.S. patent Ser. No. 12/113,672, filed on May 1, 2008, the contents of which are entirely incorporated herein by reference.

In one example, a lower concentration of the cross-linking agent is broadly applied to the cornea, e.g., in the form of a drip, after a pattern of energy is applied. The lower concentration of the cross-linking agent is effective in activating cross-linking in the regions treated with energy, because these regions receive more cross-linking agent. Meanwhile, the effect on the other regions of the cornea may be insignificant due to the low concentration and the lower amount of cross-linking agent received by these other regions.

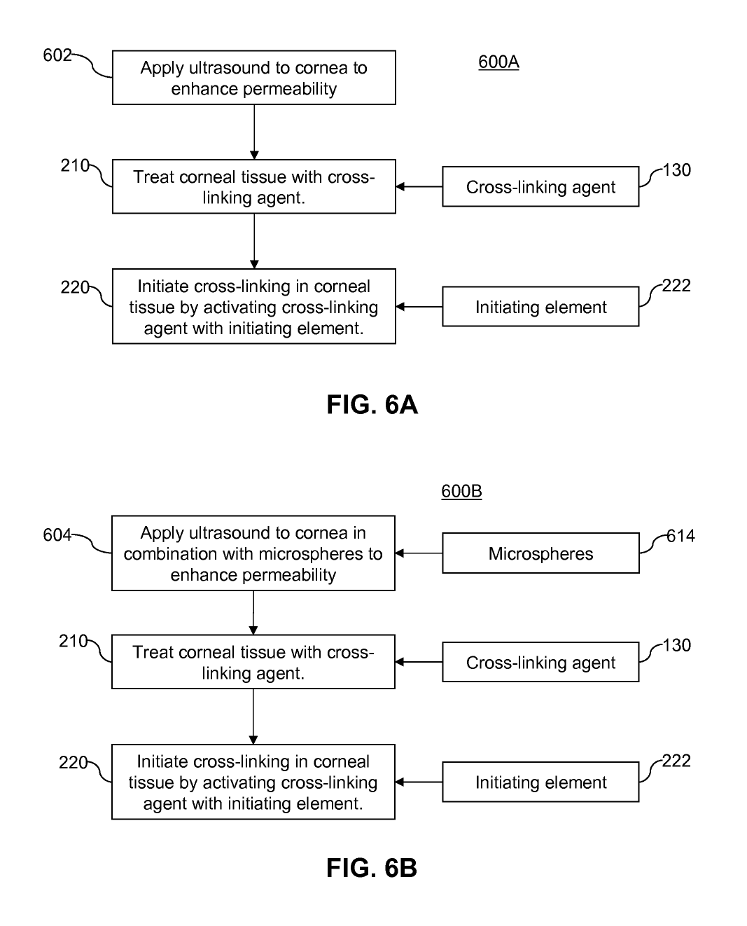

Referring to the embodiment 600A shown in FIG. 6A, ultrasound is applied to the cornea 2 in step 602 before the cross-linking agent 130 is applied in step 210. Ultrasound produces minor structural changes in the epithelium 2A and enhances corneal permeability to the cross-linking agent 130. In one example, the ultrasound may be applied at a frequency of about 880 kHz with intensities in the range of about 0.19 to 0.56 W/cm² in a continuous wave for about five minutes. However, it is understood that the ultrasound may be applied according to other parameters to achieve the desired level of corneal permeability. For example, permeability increases with increasing ultrasound intensity. In addition, the ultrasound may be delivered, alternatively or additionally, in a pulsed wave. Furthermore, the cross-linking agent 130 may be combined in varying concentrations with another agent, such as EDTA, benzalkonium chloride, or an alcohol, to promote further delivery across the epithelium 2A.

Referring to the embodiment 600B shown in FIG. 6B, the effectiveness of the ultrasound may be enhanced by applying the ultrasound in combination with microspheres (or microbubbles) 614 as is shown in step 604 of the embodiment 600B. The microspheres 614 measure approximately 1 to 3 μm in diameter. In some embodiments, the microspheres 614 may be perflutren lipid microspheres, i.e., lipid coated spheres filled with octafluoropropane gas. The microspheres 614 are sufficiently small and stable to transition into the cornea 2. The microspheres exhibit lower acoustic impedance and allow the ultrasound energy applied to the cornea to be more effectively focused. As such, the ultrasound energy applied in step 604 may be reduced relative to the level applied in step 602 of embodiment 600A, while producing the desired increase in permeability of the cornea 2.

While the embodiments 600A and 600B each provide for increasing the permeability of the corneal tissues, in some embodiments, the cross linking agent 130 may be dissolved in a different carrier to promote delivery across the corneal surface 2A. For example, the cross-linking agent 130 may be combined in varying concentrations with another agent, such as EDTA, benzalkonium chloride, or an alcohol, to promote further delivery across the corneal surface 2A.

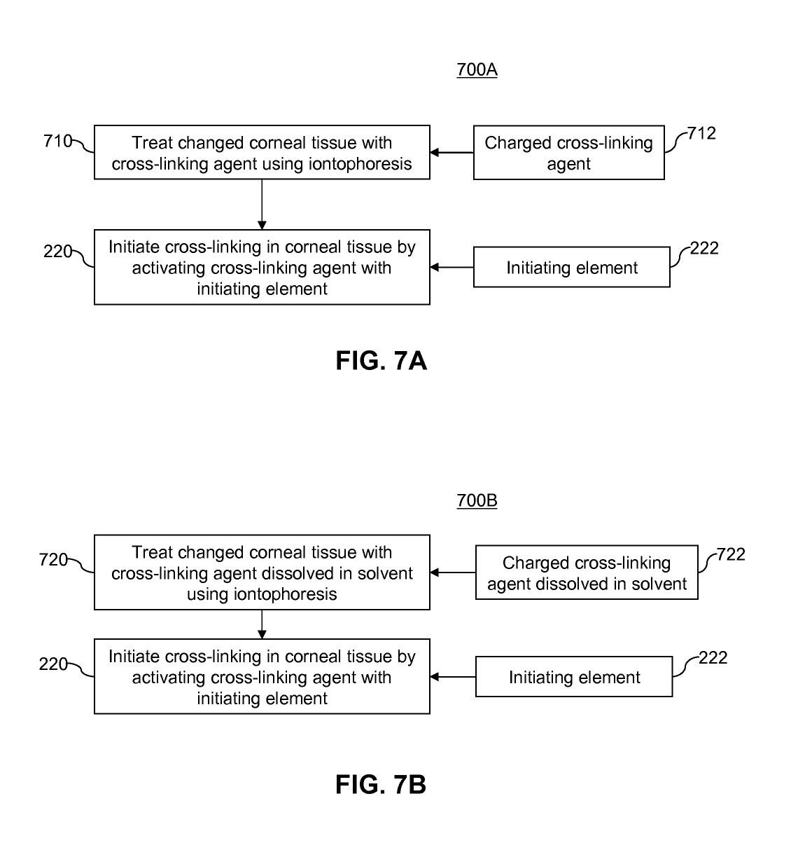

Referring to embodiment 700A shown in FIG. 7A, iontophoresis is employed in step 710 to deliver a charged cross-linking agent 712 through the epithelium 2A to the corneal tissue below. In general, iontophoresis uses an electric charge to deliver the charged cross-linking agent 712 across the epithelium 2A. High concentrations of a charged cross-linking agent 712 may be delivered transdermally by repulsive electrical forces using a small electrical charge applied to an iontophoretic chamber containing the similarly charged cross-linking agent 712. The electrical force may be an electrostatic force or an electromotive force and may be applied according to continuous waves, pulsed waves, or any combination thereof.

Referring to embodiment 700B shown in FIG. 7B, iontophoresis can be utilized to deliver a charged cross-linking agent dissolved in a solvent 722, which is shown in step 720. In the embodiment 700B, the iontophoretic chamber is filled with a solution containing the charged cross-linking agent 712 and its solvent. The solvent can be, for example, EDTA, benzalkonium chloride, or an alcohol, to promote further delivery across the corneal surface 2A.

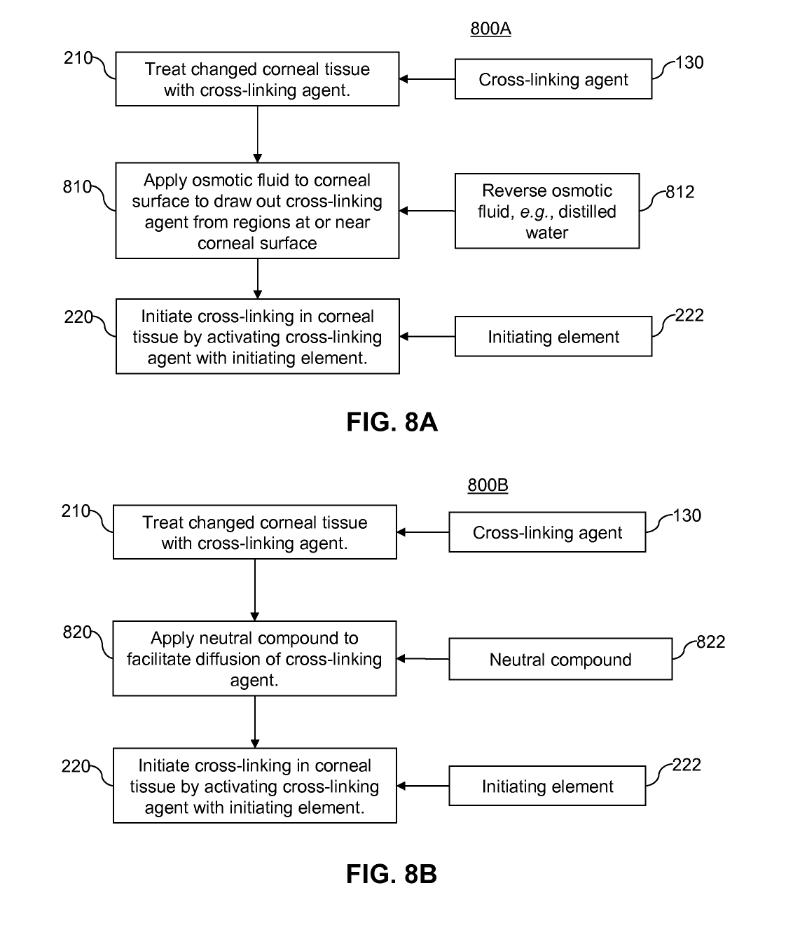

Referring to the embodiment 800A shown in FIG. 8A, the amount of the cross-linking agent 130 at varying depths of the cornea 2 may also be controlled by applying a reverse osmotic fluid 812 to the surface of the cornea 2 to draw the cross-linking agent 130 from the corneal tissue, particularly from regions near the surface of the cornea 2A. The cross-linking agent 130 is applied topically to corneal tissue in step 210. A period of time T can then optionally be allowed to pass such that, during the period of time T, the cross-linking agent 130 diffuses from the corneal surface 2A into the underlying corneal structure according to an exponential gradient. Thus, the concentration of the cross-linking agent 130 in the corneal tissue is generally greater in regions closer to the surface (epithelium 2A). In some cases, it is desirable to have greater concentrations of the cross-linking agent 130 in regions farther below the corneal surface 2A relative to the concentrations of the cross-linking agent 130 in regions closer to the corneal surface 2A. For example, greater concentrations of the cross-linking agent 130 at greater depths may be more effective in stabilizing the corneal structure, whereas excessive cross-linking at or near the surface may create unwanted effects.

To achieve greater concentrations of Riboflavin farther below the surface, in step 810, the reverse osmotic fluid 812, such as distilled water, is applied to the corneal surface 2A. The reverse osmotic fluid 812 at the surface acts to draw the cross-linking agent 130 out from regions of corneal tissue closer to the corneal surface 2A. The concentration of the cross-linking agent 130 in the regions closer to the corneal surface 2A is then reduced relative to the regions farther below. Thus, the use of the reverse osmotic fluid 812 can produce a “reverse gradient” of the amount of the cross-linking agent 130 along a path moving deeper into the cornea 2.

The resulting distribution of the cross-linking agent 130 is then activated by the application of an initiating element 222, e.g., UV light, in step 220 to initiate cross-linking in the corneal regions treated with the cross-linking agent 130. The cross-linking activity generally corresponds to the distribution of cross-linking agent 130, which is produced in part by the application of the reverse osmotic fluid 812 in step 810.

Referring to the embodiment 800B shown in FIG. 8B, a second (neutral) compound 822 may be applied after the cross-linking agent 130 is applied in step 820. The second compound 822 applies a pressure to the cross-linking agent 130 and promotes diffusion of the cross-linking agent 130 to depths of the cornea 2. The neutral compound 822 may be applied to the cornea 2 at any time during the embodiments described herein. For example, the second compound 822 may be applied at a time when diffusion of the cross-linking agent 130 has slowed and needs to be encouraged by the neutral compound.

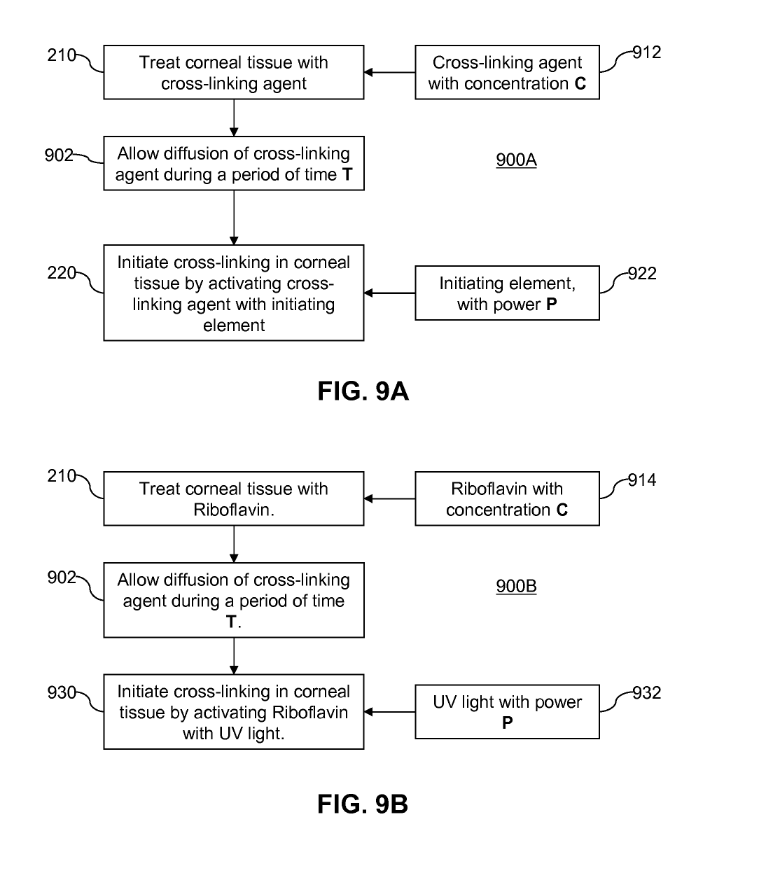

FIGS. 9A and 9B provide approaches for conveying a cross-linking agent to a region of the cornea 2 by controlling the concentration of the cross-linking agent and the time allowed for the cross-linking agent to diffuse before it is initiated.

Referring to FIG. 9A, an example embodiment 900A according to aspects of the present disclosure is illustrated. Specifically, in step 210, corneal tissue is treated with a cross-linking agent 912 with a concentration C. Step 210 may occur, for example, after a treatment is applied to generate structural changes in the cornea 2 and produce a desired shape change. The cross-linking agent 912 may be applied to the regions of the corneal tissue where the structural changes have occurred and/or to areas around the structural changes. The cross-linking agent 912, for example, may be applied topically to the epithelium 2A of the cornea 2. In step 902, a period of time T is allowed to pass. During the period of time T, the cross-linking agent 912 diffuses into the underlying corneal structure according to an exponential gradient. The distribution of cross-linking agent, i.e., concentration of cross-linking agent at depths at and below the epithelium, depends at least on the concentration C and the period of time T.

The cross-linking agent 912 is then activated in step 220 with an initiating element 922. Activation of the cross-linking agent 912 is triggered by the application of microwaves or light. As such, the initiating element 922 is applied with a power P. The power P of the initiating element 922 determines the extent to which the distribution of cross-linking agent 912 is activated. For example, an initiating element applied with a greater power P may reach greater depths below the epithelium 2A and allow the cross-linking agent to be activated at these depths. The parameters C, P, and T may be selected as independent variables to achieve the appropriate amount of cross-linking at desired depths of the cornea.

As the example embodiment 900B of FIG. 9B shows further, Riboflavin may be applied topically as a cross-linking agent 914 to corneal tissue in step 910. As also shown in FIG. 2, ultraviolet (UV) light is applied as an initiating element 932 in step 930 to initiate cross-linking in the corneal areas treated with Riboflavin. Specifically, the UV light 932 initiates cross-linking activity by causing the applied Riboflavin 914 to release reactive oxygen radicals in the corneal tissue. In particular, the Riboflavin acts as a sensitizer to convert O₂ into singlet oxygen which causes cross-linking within the corneal tissue. According to one approach, the Riboflavin may be applied topically to the corneal surface 2A, and transepithelial delivery allows the Riboflavin to be applied to the corneal stroma.

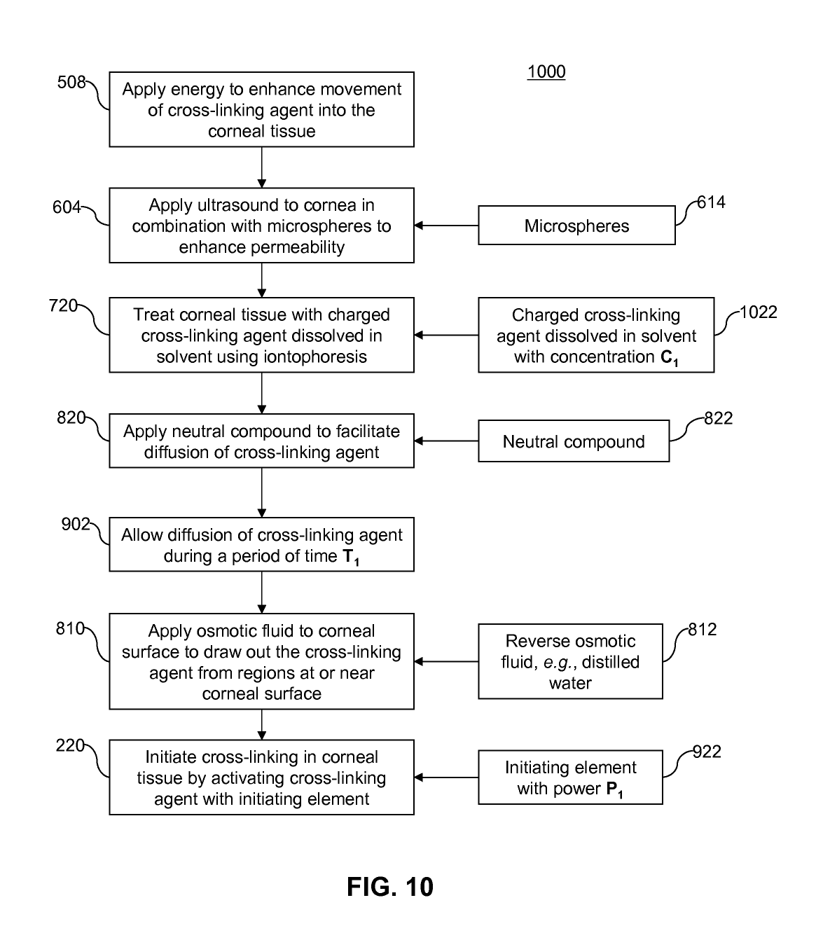

FIG. 10 provides a flowchart according to an example embodiment 1000 illustrating an approach for conveying the cross-linking agent to regions of the cornea 2 according to a combination of several different approaches previously discussed. Thus, the embodiment 1000 illustrates that the various systems and approaches discussed herein can be used in any combination or subset to precisely convey the cross-linking agent 130 to regions of the cornea 2 and thereby control the regions of the cornea 2 where cross-linking is initiated and the biomechanical strength is increased. In step 508, energy is applied to the cornea to enhance the movement of the cross-linking agent 130 into the corneal tissue (e.g., increase the permeability of the cornea 2 to the cross-linking agent). In step 604, ultrasound is applied to the cornea 2 in combination with microspheres 614 that focus the acoustic energy and enhance the increase in permeability due to the ultrasound. In step 720, the corneal tissue is treated with a charged cross-linking agent 1022 dissolved in a solvent with a concentration C₁. Also in step 720, the charged cross-linking agent is urged to penetrate beyond the epithelium using iontophoresis. In step 820, a secondary (neutral) compound 822 is applied to the eye to facilitate diffusion of the cross-linking agent through the epithelium 2A. In step 902, the cross-linking agent is allowed to diffuse into the corneal tissue during a period of time T₁. In step 810, a reverse osmotic fluid 812 is applied to the corneal surface 2A to draw the cross-linking agent away from regions at or near the corneal surface 2A. In step 220, the cross-linking agent thus distributed is activated with an initiating element 922 having a power P₁.

While the embodiment 1000 shown in FIG. 10 is shown as a flowchart, it is understood that the order of the steps as provided is not critical and many variations are possible. Furthermore, the embodiment 1000 may be further modified such that the entire procedure or portions thereof are carried out repeatedly and/or iteratively so as to gradually convey the cross-linking agent to regions of the cornea 2. In iterative implementations of the embodiment 1000, each iteration of the steps (508, 604, 720, 820, 902, 810) can be identical, can be adjusted according to predetermined or dynamically determined information, or can be omitted. For example purposes: the microspheres 614 may be applied in combination with the ultrasound treatment on the first iteration of the embodiment 1000, but omitted on subsequent iterations; the concentration C₁ of the cross-linking agent 1022 can be adjusted to a concentration C₂, C₃, . . . Cₙ on subsequent iterations; similarly, the period of time T₁ in step 902 can be adjusted to times T₂, T₃, . . . Tₙ; the power P₁ of the initiating element 922 can be adjusted to powers P₂, P₃, . . . Pₙ; the step 810 can only be applied on a final iteration; the pattern and intensity with which the energy is applied in step 508 and/or the ultrasound is applied in step 604 can each be adjusted on each iteration, the order of the steps can be adjusted from one iteration to the next, etc. In addition, steps similar to the step 902 which allow for waiting for a period of time can be added between any of the steps in the embodiment 1000 and the period of time between each step for each iteration can vary according to predetermined or dynamically determined information.

Further, in an iterative implementation of the embodiment 1000, one or more of the steps (508, 604, 720, 820, 902, 810) can be adjusted according to feedback information indicative of the progress of cross-linking in strengthening the corneal tissue. In an example embodiment, feedback information can be supplied by a feedback system configured to dynamically monitor cross-linking in the corneal tissue and provide an output signal indicative of a biomechanical strength of the corneal tissue. The feedback system can include an interferometer dynamically monitoring a three-dimensional surface profile of the surface of an eye and determining a biomechanical strength of the corneal tissue based on an amount of dynamic deformation of the surface profile of the eye due to, for example, changes in intraocular pressure corresponding to a cardiac cycle.

In sum, embodiments stabilize a three-dimensional structure of corneal tissue through controlled application and activation of cross-linking in the corneal tissue. For example, the cross-linking agent and/or the initiating element are applied in a series of timed and controlled steps to activate cross-linking incrementally. Moreover, the delivery and activation of the cross-linking agent at depths in the cornea 2 depend on the concentration(s) of the cross-linking agent and the power(s) of the initiating element.

Although cross-linking agents, such as Riboflavin, may be effectively applied to the stroma by removing the overlying epithelium before application, it has been shown that cross-linking agents can chemically transition across the epithelium into the stroma. Indeed, Riboflavin may also be delivered to the stroma by applying it topically on the epithelium. Moreover, in some cases, the epithelium may be treated to promote the transition of the cross-linking agent through the epithelium. Accordingly, in the embodiments described herein, no removal of the epithelium is required. Advantageously, this eliminates the post-operative pain, healing period, and other complications associated with the removal of the epithelium.

Although embodiments of the present disclosure may describe stabilizing corneal structure after treatments, such as LASIK surgery and thermokeratoplasty, it is understood that aspects of the present disclosure are applicable in any context where it is advantageous to form a stable three-dimensional structure of corneal tissue through cross-linking.

The embodiments above may be described with respect to treatment of the cornea and the application of a cross-linking agent. In general, however, embodiments according to the present disclosure take advantage of the increase in the permeability of eye tissue caused by the application of energy. It is further understood that aspects of the present disclosure may be employed with other eye features, such as the limbus, sclera, and retina. Thus, for example, the increased permeability may be advantageous in treating retinal membrane problems. In addition, the application of energy may be employed to produce changes in permeability in the structure of the limbus and the sclera. For example, energy may be applied to form a 6 mm to 8 mm arc at the limbus overlying Schlemm's canal. The corresponding structural changes may result in lowering intraocular pressure, for example, in the treatment of glaucoma. Increased scleral permeability without the induction of fibroblastic elements may be an effective way to lower intraocular pressure. With the increased fluid movement after energy application, significant lowering of intraocular pressure may occur. Advantageously, the amount of energy may be applied with precision.

Aspects of the present disclosure provide for lowering intraocular pressure by generating structural changes in at least one eye component of an eye. The at least one eye component may be a cornea, a limbus, a sclera, and/or a retina. According to aspects providing treatment methods for glaucoma, the cross-linking agent is not necessarily conveyed to the cornea. Aspects provide for treating glaucoma or other eye conditions by decreasing intraocular pressure with or without cross-linking also taking place. Conventional glaucoma treatments provide for decreasing intraocular pressure by regulating the flow of aqueous humor through use of, for example, prescription medications or surgical interventions. Conventional treatments to decrease intraocular pressure may decrease capillary size, or surgically redirect a flow of aqueous humor. However, aspects of the present disclosure provide for decreasing intraocular pressure to treat glaucoma by generating a structural change of an eye component by application of energy to the eye component. For example, heat energy can be applied to the eye component using the applicator 310 shown in FIG. 3.

In other embodiments, additional techniques are employed to control the amount of cross-linking agent that is delivered to the treated regions. For example, systems and methods for controlling the activation of the cross-linking agent 130 by precisely delivering the initiating element both spatially and temporally, and optionally according to information received from a feedback system are provided in U.S. patent Ser. No. 13/051,699, filed Mar. 18, 2011, and which claims priority to U.S. Provisional Application No. 61/315,840, filed Mar. 19, 2010; U.S. Provisional Application No. 61/319,111, filed Mar. 30, 2010; U.S. Provisional Application No. 61/326,527, filed Apr. 21, 2010; U.S. Provisional Application No. 61/328,138, filed Apr. 26, 2010; U.S. Provisional Application No. 61/377,024, filed Aug. 25, 2010; U.S. Provisional Application No. 61/388,963, filed Oct. 1, 2010; U.S. Provisional Application No. 61/409,103, filed Nov. 1, 2010; and U.S. Provisional Application No. 61/423,375, filed Dec. 15, 2010, the contents of these applications being incorporated entirely herein by reference. These and other techniques may be combined with the application of energy to enhance the movement of the cross-linking agent into selected corneal regions.

The use of Riboflavin as the cross-linking agent and UV light as the initiating element in the embodiments above is described for illustrative purposes only. In general, other types of cross-linking agents may be alternatively or additionally employed according to aspects of the present disclosure. Thus, for example Rose Bengal (4,5,6,7-tetrachloro-2′,4′,5′,7′-tetraiodofluorescein) may be employed as the cross-linking agent 130, or as the cross-linking agent delivered in varying concentrations 912, 1022. Rose Bengal has been approved for application to the eye as a stain to identify damage to conjunctival and corneal cells. However, Rose Bengal can also initiate cross-linking activity within corneal collagen to stabilize the corneal tissue and improve its biomechanical strength. Like Riboflavin, photoactivating light may be applied to initiate cross-linking activity by causing the Rose Bengal to convert O₂ in the corneal tissue into singlet oxygen. The photoactivating light may include, for example, UV light or green light. The photoactivating light may include photons having energy levels sufficient to individually convert O₂ into singlet oxygen, or may include photons having energy levels sufficient to convert O₂ into singlet oxygen in combination with other photons, or any combination thereof.

The present disclosure includes systems having controllers for providing various functionality to process information and determine results based on inputs. Generally, the controllers (such as the controllers 120, 140 described throughout the present disclosure) may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

As described above, the controllers may be a programmable processing device, such as an external conventional computer or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGAs), digital signal processors (DSPs), micro-controllers, and the like, programmed according to the teachings of the exemplary embodiments of the present disclosure, as is appreciated by those skilled in the computer and software arts. The physical processors and/or machines may be externally networked with image capture device(s), or may be integrated to reside within an image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described in connection with a number of exemplary embodiments, and implementations, the present disclosure is not so limited, but rather covers various modifications, and equivalent arrangements.

What is claimed is:

1. A method for activating cross-linking in at least one eye component, comprising:
delivering ultrasound waves to the at least one eye component such that a permeability of the at least one eye component to the cross-linking agent is increased;
conveying the cross-linking agent to the at least one eye component; and
activating the cross-linking agent by delivering an initiating element to the at least one eye component.

2. The method of claim 1, further comprising:
applying microspheres to the at least one eye component to focus the ultrasound waves applied to the at least one eye component.

3. The method of claim 1, further comprising:
dissolving the cross-linking agent in a solvent to facilitate penetration of the at least one eye component.

4. The method of claim 1, wherein the epithelium of a cornea is not removed.

5. The method of claim 1, wherein the cross-linking agent is delivered at a first concentration and the initiating element is delivered according to a first dose specified by a first power, the method further comprising:
conveying a second concentration of the cross-linking agent to the at least one eye component; and
activating the cross-linking agent according to a second dose specified by a second power.

6. The method of claim 1, wherein the ultrasound waves are delivered to the at least one eye component in a pattern such that some regions of the at least one eye component are more permeable to the cross-linking agent than others.

7. The method of claim 1, wherein the at least one eye component is at least one of: a cornea, a limbus, a sclera, and a retina.

8. The method of claim 1, wherein the cross-linking agent is Riboflavin or Rose Bengal and the initiating element is ultraviolet light.

9. A method for treating an eye, comprising:
delivering heat energy to at least one eye component of the eye;
generating a structural change in the at least one eye component; and
lowering an intraocular pressure of the eye due to the generated structural change.

10. The method of claim 9, further comprising:
conveying a cross-linking agent to the at least one eye component; and
activating the cross-linking agent by delivering an initiating element to the at least one eye component.

11. The method of claim 9, wherein the intraocular pressure in the eye is lowered to treat glaucoma.

12. The method of claim 9, wherein the at least one eye component is at least one of a cornea, a limbus, a sclera, and a retina.

13. A method for activating cross-linking in at least one eye component, comprising:
delivering heat energy to the at least one eye component such that a permeability of the at least one eye component to a cross-linking agent is increased;
conveying a cross-linking agent to the at least one eye component; and
activating the cross-linking agent by delivering an initiating element to the at least one eye component.

14. The method of claim 13, wherein the delivering heat energy rearranges corneal fibrils to generate a desired change in the shape of a cornea.

15. The method of claim 13, wherein the delivering heat energy does not substantially change a desired shape of a cornea.

16. The method of claim 13, wherein the epithelium of a cornea is not removed.

17. The method of claim 13, wherein the cross-linking agent is delivered at a first concentration and the initiating element is delivered according to a first dose specified by a first power, the method further comprising:
conveying a second concentration of the cross-linking agent to the at least one eye component; and
activating the cross-linking agent according to a second dose specified by a second power.

18. The method of claim 13, wherein the heat energy is delivered to the at least one eye component in a pattern such that some regions of the at least one eye component are more permeable to the cross-linking agent than others.

19. The method of claim 13, wherein the at least one eye component is at least one of a cornea, a limbus, a sclera, and a retina.

20. The method of claim 13, wherein the cross-linking agent is Riboflavin or Rose Bengal and the initiating element is ultraviolet light.

21. A method for activating cross-linking in at least one eye component, comprising:
conveying a charged cross-linking agent to the at least one eye component;
urging the charged cross-linking agent to a depth of the at least one eye component using iontophoresis; and
activating the cross-linking agent by delivering an initiating element to the at least one eye component.

22. The method of claim 21, further comprising:
charging the cross-linking agent with an electrical charge; and
dissolving the cross-linking agent in a solvent.

23. The method of claim 21, wherein the urging is carried out by generating a continuous or pulsed electromotive force that repels the charged cross-linking agent.

24. The method of claim 21, wherein the at least one eye component is at least one of a cornea, a limbus, a sclera, and a retina.

25. A method for activating cross-linking in at least one eye component of an eye, comprising:
conveying the cross-linking agent to a surface of the at least one eye component;
allowing a time to pass to allow the cross-linking agent to diffuse within the at least one eye component;
applying a reverse osmotic fluid to the surface of the at least one eye component to draw out the cross-linking agent at or near the surface of the at least one eye component; and
activating the cross-linking agent by delivering an initiating element to the at least one eye component.

26. The method of claim 25, wherein the reverse osmotic fluid is distilled water.

27. The method of claim 25, wherein prior to the activating the cross-linking agent, the cross-linking agent is distributed such that a greater amount of the cross-linking agent exists below the surface of the at least one eye component than exists at or near the surface of the component.

28. The method of claim 25, wherein the at least one eye component is at least one of a cornea, a limbus, a sclera, and a retina.
